(19) United States
(12) Patent Application Publication
(10) Pub. No.: US 2009/0099441 A1
(43) Pub. Date: Apr. 16, 2009

(54) BRAIDED ELECTRODES

(75) Inventors: Simon Giszter, Havertown, PA (US); Frank K. Ko, Philadelphia, PA (US); Ubong Ime Udoekwere, Philadelphia, PA (US); Heejae Yang, Secane, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignees: Drexel University, Philadelphia, PA (US); Philadelphia Health & Education Corporation d/b/a Drexel University College of Medicine, Philadelphia, PA (US)

(21) Appl. No.: 12/065,697

(22) PCT Filed: Sep. 8, 2006
(86) PCT No.: PCT/US2006/035028

§ 371 Date: Oct. 9, 2008

Related U.S. Application Data
(60) Provisional application No. 60/715,228, filed on Sep. 8, 2005.

(51) Int. Cl. A61N 1/05; A61B 5/042

(52) U.S. Cl. 600/377; 607/116; 29/868

(57) ABSTRACT

Electrodes for intracorporeal and other uses are provided. The invention features sterilizable, braided electrodes which are formed of or include conductive elements in electronic communication with a plurality of sites for electrical stimulation or sensing. Other active elements may be included in the braided electrodes.

### GOVERNMENT RIGHTS AND CROSS REFERENCE TO RELATED APPLICATIONS

Pursuant to 35 U.S.C. §202(c), it is acknowledged that the United States Government may have rights in the invention described herein, which was made in part with finding from the National Institutes of Health through Grant NS44564. Benefit is claimed of provisional application number 60/715,228 filed Sep. 8, 2005.

### FIELD OF THE INVENTION

The present invention is directed to electrodes for recording of and/or stimulating a nervous system of an animal. The invention provides electrodes for other biomedical uses as well.

### BACKGROUND OF THE INVENTION

Polymeric fibrous materials are the fundamental building blocks of living systems. Fibers exist, for example, within all cells, as DNA, cytoskeleton filaments, and as cellular structures of sensory cells, such as hair cells and rod cells of the eyes, fibers form the intracellular matrices and extracellular matrices for tissues and organs. Junctions between these excitable cells conduct electrical and chemical signals to elicit various kinds of stimulation. The signals direct normal functions of the cells such as energy storage, information storage and retrieval and processing in the cells of the nervous system, tissue regeneration, and sensing.

Current methodologies use electrodes to record or stimulate these activities in the central and peripheral nervous systems. Wiring and connectors are useful in such applications; however, mechanical impedances, for example, wire tangling and knotting, are a major source of failure and neural damage; stresses on neural tissues at wire joints and junctions also contribute to neural damage. Moreover, the limited compliancy and general rigidity of current multi-electrode materials creates a risk of neural damage during accelerated motion. As current applications of these electrodes becomes more common in clinical applications, the mechanical environments to which devices are subject will likely become increasingly challenging.

In addition, many current electrodes generally allow for only low recording densities, providing limited information or stimulation. Moreover, current materials and methods allow for only limited modulation of the physical, mechanical, and conductive properties of present electrodes. Thus, what are needed are more flexible materials with alterable physical properties.

### SUMMARY OF THE INVENTION

The present invention provides electrodes which are suitable for implantation in vivo and otherwise. In accordance with preferred embodiments, the electrodes are sterilizable such that their use for sensing electrical potential in the body of a subject may be performed. Additionally, such electrodes are useful for the delivery of electrical signals or stimuli to such subjects. The electrodes of the invention are especially useful in the environment of the brain or otherwise in the nervous system of a patient. Electrodes of the invention comprise a braid and have a plurality of electrical conductors. A plurality of sites on the electrode are defined for sensing or stimulation and at least some of the conductors are in electrical communication with the sites. The stimulatory/sensing sites may be caused to exist in a geometrically defined patent and in a relatively high density.

### BRIEF DESCRIPTION OF THE DRAWINGS

FIGS. 1a-1c provides partial schematic views of an exemplary embodiment of a microbraided electrode of the invention.

FIGS. 2a-2c depict recording / stimulating sites on a braided electrode. FIGS. 3a and 3b depict an embodiment of the invention featuring certain biodegrading elements which dissolve or degrade in vivo leaving areas of exposed conductor behind on non-biodegradable elements.

FIGS. 4a and 4b show conductor elements surrounded by a biodegrading braided element. Upon dissolution or degrading of the element, the conductors gently expand in geometry.

FIG. 5 shows in schematic an array of electrodes in cooperation with a support.

FIG. 6 illustrates the introduction of an array of electrodes into the brain of a subject.

FIG. 7 shows the elaboration of a flat braid electrode in accordance with the invention. In the depicted embodiment, a spiral version of a “Chinese finger trap” arrangement can be formed. FIG. 7b shows the attachment of the electrode around a nerve periphery. Flat surfaces (“Flat interface neural electrode” FINE) of flat or figured braided conductors can be constructed in a similar fashion.

In addition to use in neurological contexts, the electrodes of the invention may also be used in medical contexts apart from the brain or nervous system such as in the delivery of stimulatory signals for tissue regrowth, for stimulation of other sorts and in ex vivo applications. The present electrodes may find use in industrial applications as well, especially where pluralities of sensing or stimulatory sites are required in predefined, spatially organized fashions.

The braided electrodes of the invention may feature any braided structure, tubular, flat, figured or more complex braids being suitable and known per se. Braided structures which may have their geometries altered in preselected and predictable ways are also featured. Employment of different patterns of stimulation/sensing sites may be performed with one pattern being used for sensing and another for stimulation. Alternatively the patterns may overlap. It is not necessary that one braided electrode perform both sensing and stimulation, however.

The conductors may form part of the braid, may be “laid into” the braid or both. A conductor which is “laid into” a braid is one which is ensnared by the braid, but which does not form part of the braid itself. In some embodiments, some or all of the conductors are monofilaments. In some embodiments, at least some of the conductors have lengths which are at least about 100 times their diameters. In other embodiments, conductors have average diameters on the order of from about one μm to about 50 μm. In others, conductor average diameters on the order of from about 0.1 μm to about 1 μm are preferred. As will be appreciated, control of conductor size and geometry permits the careful control of the geometry and spacing of the sites of stimulation and or sensing. In most cases, it is preferred that substantially all of the conductors have average diameters less than about 50 μm.

The conductors may be metal, conductive polymer, conductive protein, or conductive nanostructure, especially nanotubes or nanofilaments. All these materials are known per se.

In some embodiments, doped conjugated polymers such as polyanaline are employed in filamentous form. Carbon nanotubes are featured as conductive elements on other embodiments.

For some applications, use of materials which have geometric “memory” such as memory metals or memory polymers may be useful as the same may permit improved contact with certain body parts, conformation to complex structures and the like. The conductors are preferably insulated from each other and from the environment. Insulative materials such as PTFE, parylene-C or other otherwise inert materials are useful.

Coating of some or all of the conductors or other portions of the electrode may also be achieved to good effect. Coating with metals, such as gold, platinum, silver, iridium, other metals or combinations thereof may find use in some applications. Sputtering is a convenient way of achieving such coating although other means may also be employed such as reductive deposition and the like.

The braided electrodes of the invention may also comprise one or more optical elements, such as fiber optic strands or cables. Such inclusion may facilitate placement of the electrode or may be used in monitoring either the electrode or a body state in a subject. The present invention is particularly useful in conjunction with cannulas, catheters, probes, or other surgical or medical devices. For example, application of an electrode of the invention on or with a surgical probe facilitates the sensing of electrical potential, pH, or any of a number of body states such as glucose concentration, viscosity, or other properties adjacent the probe. Pluralities of electrodes may be arrayed on a single instrument either of the same or of different constitution.

Other active elements may be included in braided electrodes of this invention. Thus, quantum dots may be arrayed within such electrodes to report upon one or more body states in organisms or tissues into which the electrodes are introduced. The conductors may be used to transfer signals from the quantum dots to a sensing or recording device for interpretation and storage. Circuits, chips, electronic elements such as triodes, diodes, tetrodes and the like, MEMS and other elements known per se, may also be included in this way. The use of partially or wholly hollow of shaped conductors may benefit these embodiments and other aspects of the invention. Light emitting diodes, especially those coupled to a body state sensing system are particularly useful, especially when arrayed consonant with the pattern of sites for sensing or stimulation.

In one class of embodiments, braided electrodes are constructed including one or more biodegradable or dissolvable elements. If some or all of the braided materials can be caused to be completely or partially dissolved or degraded in a predictable fashion, usually after implantation, the remaining elements of the electrode may exhibit beneficial properties or results. For example, dissolution of a braid may effect the exposure of stimulation/sensing sites on conductors forming part of an electrode. Additionally, a dissolved or degraded braid may liberate conductors to assume an altered or different shape or geometry. In such a way, particularly intimate contact between conductor and tissue may be achieved. Removal of degradable material may also facilitate the long term placement of electrodes by diminishing the overall size of electrodes and by possibly improving their biocompatibility.

The biodegradable materials useful for these embodiments include vicryl, sucrose, dextrose, carbowax, mannose, polyethylene glycols, polylactic acids, polyvinyl alcohols, and any other material which can be used to elaborate braided electrodes in accordance herewith and which, at a predictable point in time or in a predictable environment, degrade or dissolve to give rise to an altered, but useful electrode.

The present invention also provides methods of manufacturing sterilizable electrodes. These comprise braiding a plurality of fibers onto, over or within a braiding form to form a braided structure. At least some of the fibers so braided are conductive and are modified so that at least some of the conductive fibers are exposed to the environment to form sites of sensing or stimulation. The fibers must be biocompatible at least to as useful degree. Other means of forming electrodes useful for the methods of the present invention may also be employed.

In accordance with other embodiments, conductors are also laid into the braid of the braided electrodes to be formed. In other embodiments, the geometries or compliances of the electrodes are altered post initial formation either by mechanically altering the shape of the braid or by causing the dissolution or degradation of some elements of the braid or otherwise. Inclusion of memory metal or memory polymer within the braids is also contemplated herein.

In some embodiments, the braid and the form upon which it is formed are used together. The braid may be mechanically, chemically or physically attached or associated with the form to achieve this purpose. Good effect may be obtained by providing that the form is conductive. It may also be degradable of soluble in biological systems.

### DETAILED DESCRIPTION OF ILLUSTRATIVE EMBODIMENTS

The present invention provides for sterilizable electrodes useful in the recording of or stimulation of the central and peripheral nervous systems. The insulated electrodes described herein are capable of sensing or stimulating at a plurality of sites along the dimensions of the electrodes. In addition, the electrodes of the present invention are comprised of a plurality of electrically independent conductors, interwoven either to form or to be incorporated within a braid. The sensing and/or stimulating sites can be on the surface of the braid or within the braid. The nature of the braided configuration has been found to allow for electrodes that are self-stabilizing, strong, and flexible and which have adjustable mechanical properties. Braided electrode construction in accordance with this invention permits the fine adjustment of electrical properties of the electrode; dimensions of exposed tips; insulation; structural integrity; and operating environment characteristics.

The braided electrodes of the present invention may be arranged in any braided configuration. The mechanical properties of these electrodes can be manipulated depending on the type of braided arrangement selected. In certain embodiments, the braids may be tubular, for example, a “Maypole dance” arrangement or a “Chinese finger trap” arrangement. In a “Chinese finger trap” arrangement, the exertion of axial force on the braid tightens the braid. Exertion of an inward force may loosen the braid.

In other embodiments, the braids may be flat. In yet others, the braids may be rectangular. In still others, the braids may comprise a figured arrangement in which the braid configuration differs along the length of the electrode. Both two-dimensional and three-dimensional braids are contemplated as being within the scope of the present invention.

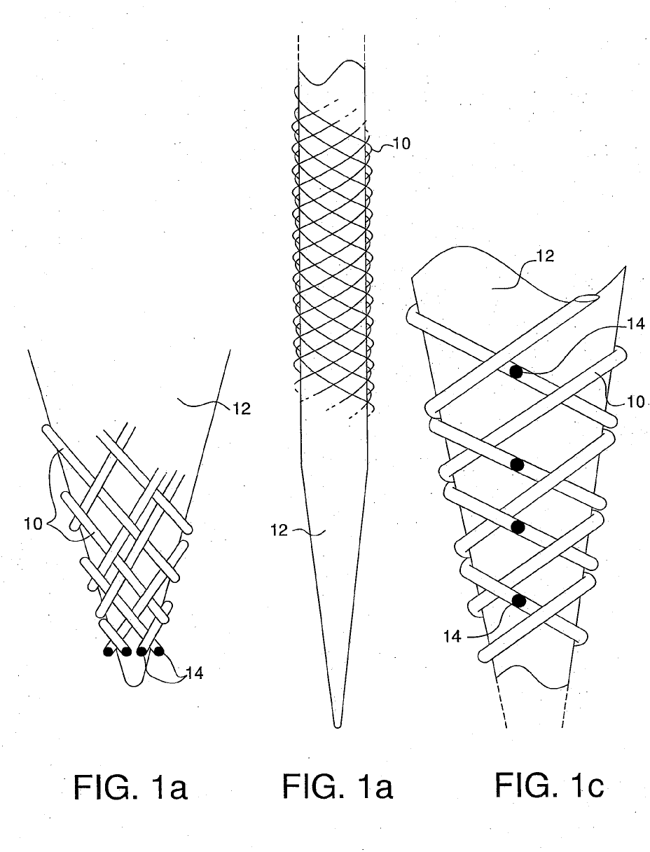

FIGS. 1a through 1c depict relatively simple braided electrodes (Maypole braid) in accordance with some embodiments of this invention. Fibers 10 here, preferred conductive fibers, are braided together over a braiding form 12. FIG. 1a shows a typical, conical structure for the tip of the electrode. It also shows sites on the conductive fibers 14 which are exposed to the environment and useful for sensing electrical potential, for achieving electrical stimulation or both. These are regularly arrayed in a geometric pattern defined by the braid. In this embodiment, the braiding form may also be active. For example, the form may be a fiberoptic device, a cannula, a micropipette or other element, which is itself useful intracorporeally. Conveniently, one sensing/actuation site per conductor is provided, although multiple sites per conductor may be useful in some embodiments. While in this figure, each filament or fiber 10 is depicted a being a single conductor, insulated except for at the activation/sensing site, each filament or fiber may also comprise pluralities of individual conductors or other fibers, fibrils or filaments. In such cases, increased density of conductors and actuation/sensing sites may be achieved.

FIG. 1b shows how a tubular braided electrode may be made by braiding fibers 10 over braiding form 12. Change in the geometry of the electrode may be achieved through appropriate shaping of the form, here into a pencil—like shape. It is also useful to form the braided electrode into a simple tubular configuration for many embodiments, whereupon use of a conical section is not made. FIG. 1c depicts a further conical braiding arrangement of electrode tip with stimulation/sensing sites 14 arrayed generally longitudinally along the braid.

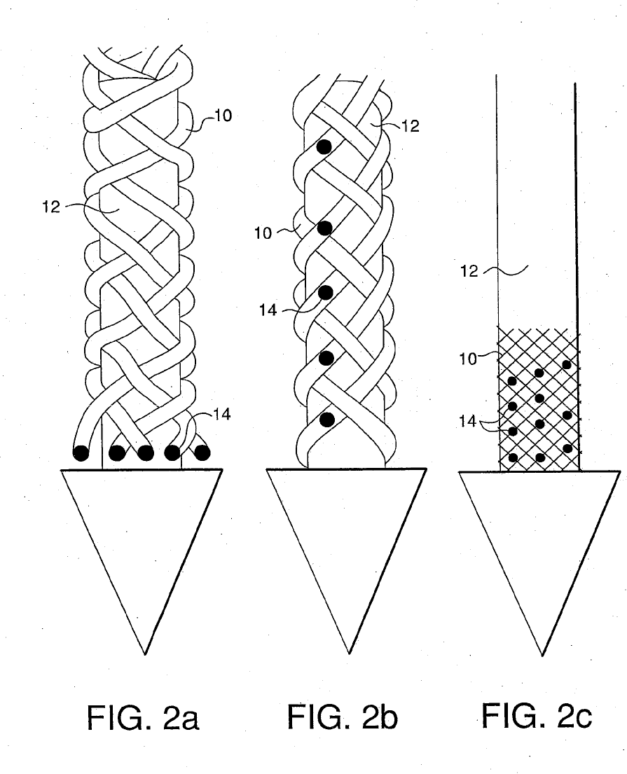

FIGS. 2a, 2b and 2c show three embodiments of the invention, each generally tubular in braid structure. The figures depict different arrangements and geometries of sensing/stimulation sites on the braids. While it is useful to have one site in electrical communication with one conductor, single conductor may communicate with pluralities of sites or vice versa. The figures are intended to imply that the braided electrodes may be removed from the braiding form after formation and used independently and in different configurations. Such release may be performed after insertion intracorporeally or otherwise.

FIG. 3a is of a tubular braided electrode comprising conducting, biologically stable filaments 22 and biologically soluble or degradable filaments 20 braided together over a form 12. As shown, the two types of fibers are braided clockwise and anticlockwise. In this case, the braided electrode, formed of both degradable or soluble fibers and non degradable or insoluble fibers, can be removed from the braiding form after braiding. After expo sure to biological conditions or to solvent, stable filaments 22, most or all of which include conducting elements, are released and form a new geometry, here a helical pattern. FIG. 3b depicts this along -with the stimulation / sensing sites 24. The geometry may further change, as shown, by relaxing, or otherwise. In this way, relative small, loosely organized conductors may be delivered to a biological situs in a relatively rigid, structured form and released to assume a relative loose, form. This arrangement permits delivery of very small electrode units and ones having minimal impact upon the organism into which the electrodes have been implanted.

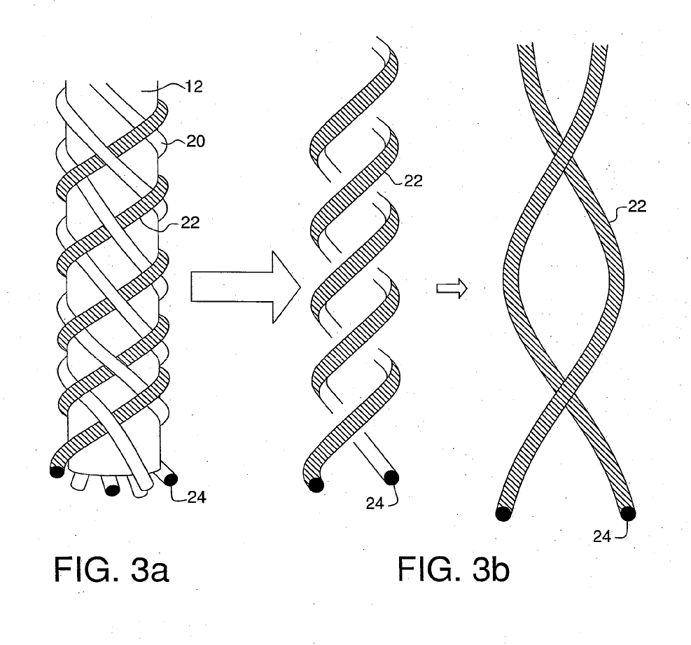

A different variation employing soluble or degradable fibers is shown in FIGS. 4a and 4b. A figured braid, formed of soluble or biodegradable fibers 20 surrounds conductive elements 22, such as nano or microscale insulated conductors or wires, at least an end portion of an electrode. Stimulation or sensing sites 24 are also shown. Here, optionally, a portion of the electrode 30, away from the distal end, the end intended for contact with tissue, is braided in a different fashion, protected or otherwise kept generally intact for purposes of improved handling. Perforce, the braiding of the electrodes may change along the electrodes' length for this and other purposes. Upon dissolution or degradation of the surrounding braid, the bundled conductors, here shown in hatching along with other, non-hatched elements, may ease apart to confer a different configuration. In some cases, improved intimacy with tissue may be had as may a lowering of strain within the group of bundled elements. In addition to the conductors, the elements of the bundle may include fiber optic, electronic, MEMS, OLED and a host of other active elements, known per se.

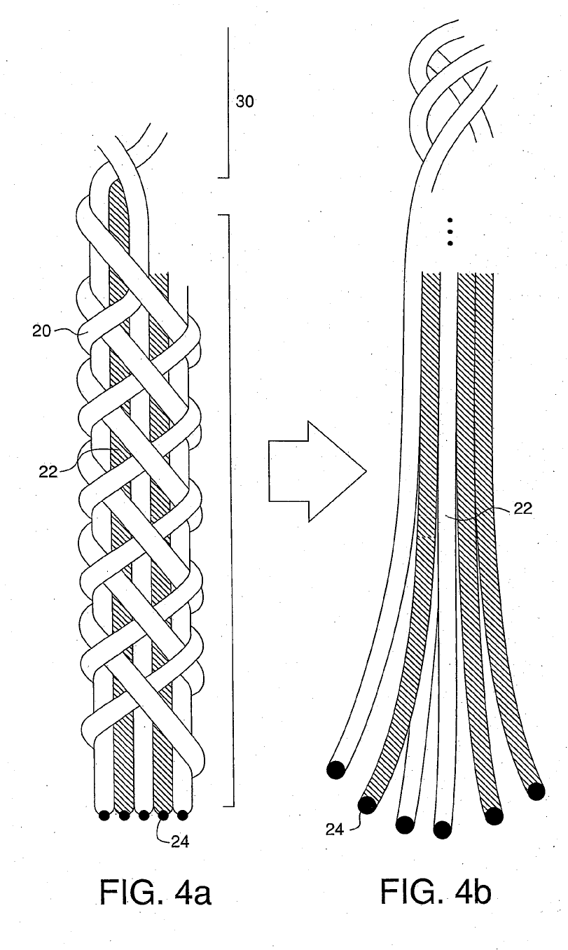

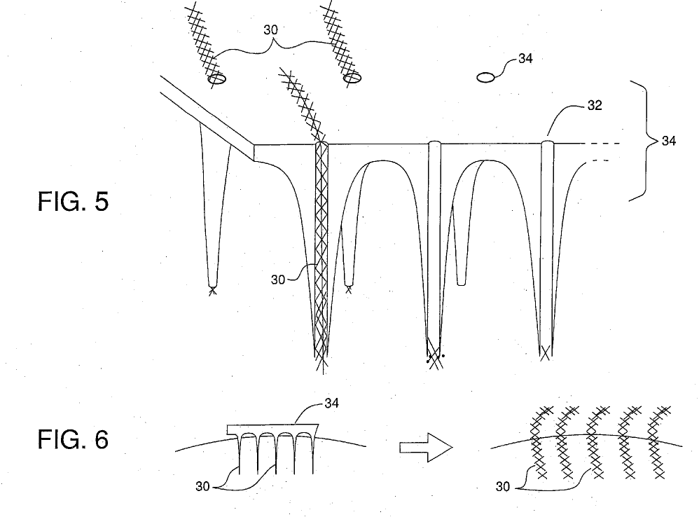

FIG. 5 depicts a spatial array 34 of electrodes 30 in accordance with this invention oriented inter se in space by a supporting or matrix element 32. Orifices in the matrix element 34 facilitate this arrangement. FIG. 6 shows the array implanted into tissue. If the matrix element is biodegradable or soluble, a spatially arrayed set of electrodes may be inserted into and caused to remain in the tissue while the matrix element is dissolved or degraded away.

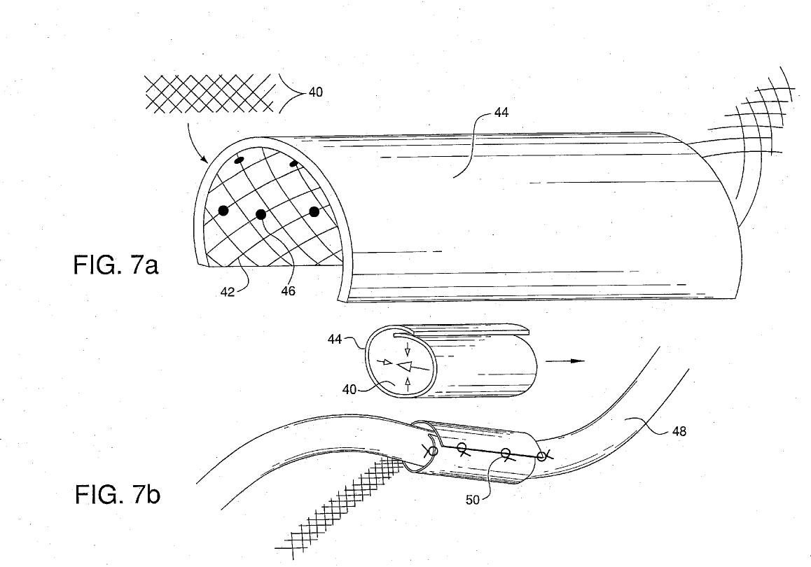

A flat braided or figured multi—electrode 40 comprised of filaments 42 is shown elaborated upon a flat, albeit curved, form 44. Electroactive—sensing or stimulating—sites 46 are formed in the braid. The resulting braided electrode may either be used as a fabric or may be used together with the form as shown. It may also be laminated or associated with biocompatible fabric, polymer or other material. The electrode assembly may be caused to surround a tissue of interest, in FIG. 7b, a peripheral nerve, and affixed in place with adhesive, with suture 50 or otherwise. Stimulation of the nerve or sensing of its electrical potential or both may thus be accomplished. A wide range of applications, such as supradural, suprapial, or subcutaneous recording sheets with or without an insulating sheet may be easily accomplished. The compliance and shape of the electrodes described herein are alterable. These properties can be altered, for example, by the selection of the materials comprising the electrodes. These properties can also be altered by altering the diameter of the materials comprising the electrodes. These properties can also be altered by altering braid configuration or topology during construction or altering braid angle and relation to the supporting and embedding materials around the braid in-situ.

The braided electrodes of the invention include conductors, which may be monofilaments, multifilaments, or other forms. In certain embodiments, at least one conductor forms a part of the braided electrode. In others, the conductor can be laid into the braid.

The conductors of the present invention may be comprised of any number of individual conductive elements. For example, the conductors may comprise metals, such as nichrome or stainless steel. They may also comprise conductive polymers such as lithium doped polyaniline and polyethylene dioxythiophene. In some embodiments, the conductors may comprise conductive proteins. In yet others, the conductors may be conductive nanotubes or nanofilaments, for example, carbon nanotubes or nanowires. These materials may be microscale, nanoscale, or combinations of both microscale and nanoscale materials. In some embodiments, the conductors may be hollow. In preferred embodiments, at least one conductor has a length that is at least 100 times greater than its diameter and, in some embodiments may be monofilaments.

The conductors are preferably insulated with a material such as with Teflon or Parylene C. In exemplary embodiments of the present invention, the conductors may comprise intermittent insulation along the length of the conductors, providing a plurality of sites along the length of the braid structure for use in sensing or stimulation of the central or peripheral nervous system.

In addition to comprising conductive elements, the braided electrodes of the present invention may further comprise biocompatible materials that can enhance the mechanical and/or electrical properties of the present invention. In preferred embodiments, these materials can be used to alter the compliance and/or shape of the braided electrodes. These materials may be nanoscale, microscale, or combinations thereof. For example, these fibers can have diameters ranging from about 600 nm to about 1000 μm (microscale fibers) or less than 600 nm (nanoscale fibers, under NSF definitions). These biocompatible materials can also be protein fibers or synthetic polymers. For example, in certain embodiments, protein fibers such as fibroin, including Bombyx mori and spider silk, and keratin, such as wool, may be incorporated into the braids of the present invention to provide mechanical strength to the braided electrodes. In other embodiments, collagen or elastin fibers are incorporated into the present invention to provide mechanical strength to the electrodes of the present invention. In is also envisioned that combinations of fibroin, keratin, collagen, and elastin may be incorporated into the present invention.

In certain embodiments, these materials comprise shape memory polymers or shape memory metals. These for example can be used to actuate braid shape changes.

Synthetic polymers may also be incorporated into the braids of the present invention to enhance the mechanical and/or electrical properties of the present electrodes. For instance, biocompatible poly-L-lactic acid can be electrospun into fibers with diameters ranging from about 150 nm to about 550 nm. Inclusion of poly-L-lactic acid into the braided electrodes can improve the mechanical properties of the electrodes by increasing the modulus. Verification of incorporation of the poly-L-lactic acid fibers into the braided electrodes can be performed using Raman spectroscopy. Polyethylene oxide (PEO), polyaniline (PANi), and blends thereof can also be electrospun to form fibers that can be incorporated into the present invention.

Certain conductive polymers having desirable mechanical properties, for example, PEDT, can also be incorporated into the braided electrodes described herein. Other desirable polymers include, but are not limited to, polylysine, a blend of polyethylene oxide/polyaniline, polyacrylonitrile, poly (3,4-ethylenedioxythiophene) poly(styrenesulfonate), a blend of poly(3,4-ethylenedioxythiophene)/ polyacrylonitrile, or polylactic-co-glycolic acid.

In some embodiments, the braided electrodes of the present invention may further comprise biodegradable materials. In such embodiments, certain biodegradable materials, for example vicryl, sucrose, dextrose, carbowax, mannose, or polyethylene glycol, may be incorporated into the braid to enhance its mechanical properties. For example, the biodegradable material might impart stiffness to the braid, facilitating the insertion of the electrode to the desired site. Once inserted into the nervous system, the material would then biodegrade, resulting in an in situ modification of the mechanical properties of the electrode. For example, once the material biodegrades, the electrode would usually become more flexible, and/or alter spatial shape.

In other embodiments, dissolvable, for example, materials that dissolve in solvents ex vivo, may be incorporated into the braided electrodes. In certain embodiments, such materials could be incorporated into the electrodes during their manufacture to provide certain mechanical properties to the electrodes. After manufacture, the electrodes could be immersed in a suitable solvent to dissolve the dissolvable material, thus altering the mechanical properties of the electrodes prior to insertion at the desired situs.

Electrodes of the present invention may further comprise quantum dots. A quantum dot is a semiconductor nanostructure that confines the motion of conduction band electrons in three spatial directions. Electrodes of the present invention may also comprise microelectromechanical systems within the braided structure.

In preferred embodiments, the electrodes of the present invention form at least one triode. In others, the electrodes form at least one tetrode. In still others, electrodes of the present invention form at least one polytrode. Electrodes described herein may also exist as part of a composite structure.

Electrodes of the present invention may also comprise a plurality of individual braided electrodes. In some embodiments, several braided electrodes can be assembled to form an array of braided electrodes arranged in rows and/or columns like tines of combs. Preferably, the electrodes can be assembled to form a cuff electrode suitable for placement around a nerve.

It is also envisioned that electrodes of the present invention may further comprise cannulas, catheters, or other surgical devices through which fluids could be delivered to the situs of the electrode.

In certain embodiments, a plurality of independent conductors can be “laid into” the braid. As used herein, “laid into” refers to the incorporation of a material into the braided structure. In other embodiments, these conductors can be modified by exposing at least some of them to the environment. Preferably, the compliance and/or shape of these braided electrodes can be altered in a controlled fashion.

Electrodes of the present invention may be formed by braiding a plurality of fibers onto, over, or within a braiding form, wherein some of these fibers are independently conductive. A braiding form is any material upon which materials can be braided. All braiding configurations are envisioned. The fibers can be modified by exposing them to the environment. This exposure can take place along the length of the braided electrode, at a plurality of sites. The fibers should be biocompatible and able to withstand sterilization conditions.

In some exemplary embodiments, the braiding form remains within the braided electrode. In some embodiments, the braiding form is inert to environmental conditions. In others, the braiding form is biodegradable or dissolvable. In still others, the braiding form has shape memory properties. In yet others, the braiding form is conductive. For example, the braiding form may comprise a fiberoptic material such as quartz. The braiding form may also comprise a metal such as tungsten. In some embodiments, the braiding form comprises glass. In other embodiments, the compliance of the braiding form is alterable.

In a preferred embodiment, the braided structure is affixed to the braiding form. For example, the braided structure can be affixed to the braiding form with an adhesive such as cyanoacrylate. In some embodiments, the braided structure can be affixed to the braiding form with biodegradable or dissolvable materials, for example, mannose.

In one embodiment, the braiding form may comprise a fiberoptic fiber. Such embodiments may be useful in, for example, focal uncaging of caged neurotransmitters. In preferred embodiments, the fiberoptic fiber is sputter coated with a metal, for example, platinum, for recording. Preferably, the fiberoptic is insulated. Exemplary examples of insulating materials are Parylene-C and PTFE.

In one exemplary embodiment of the present invention, the braiding form is a fiberoptic quartz fiber, preferably 50 μm. This fiber is pulled to a fine tip, for example 10 μm. Caged compounds can be delivered into the cord via a tubular braid or a cannula of carbon nanofibers or other very fine fibers from different polymers. The nanofibers are braided to form a 30 μm shaft and coated with medical grade silicon rubber. A guide provides the cannula stiffness for insertion into the spinal cord, and can be removed once the cannula is in place. The cannula left in place is highly flexible, thus limiting damage to the spinal cord. The conductive filaments can be spaced precisely and they can be separated by nonconductive filaments of finer diameter.

To assure precision fiber placement and facilitate the manipulation of very delicate micron level filaments, a computer aided design and manufacturing system capable of nanoscale and microscale fiber placement can be used. For this system, a geometric design algorithm can be developed and the information translated to machine instruction code. To provide sufficient structural stability, it is preferred that a computer controlled microbraiding system be used. Such a system is extendible to nanoscale levels by the use of a nanomanipulator system such as the Zyvex L100 nanomanipulator capable of operating in an SEM chamber.

One exemplary embodiment for this braider is a hexagonal braiding machine wherein the fiber carriers travel in a gradual path. This system is capable of moving more filaments thus resulting in high filament density. The microbraiding system may have a unique hexagonal cam of 1.75 inch diameter. Each cam can carry up to three bobbins for braiding thus providing flexibility in the numbers of filaments used. Each cam can be individually controlled by a dedicated stepper motor for more precise and gentle control.

The design and fabrication of components of the braider can be carried out by: using a CAD/CAM system. The parts can be designed using CAD software, for example, Solidworks 2004. Cams and bobbins can be fabricated using a rapid prototyping equipment SLA, for example, Stereolithography Apparatus, 3D systems series7. The prototype cams and bobbins can be made of UV curable epoxy resin. The braiding frame and other structural components can be fabricated using CAM (Bridgeport Interact 720). The smallest functional unit for this type of braider consists of six cams and can accept up to twelve bobbins. This functional unit can be expanded easily to a 3-dimensional braider for complex 3-D braiding of 3-D electrodes by adding more cams around it. The stepper motors are connected to external driving circuits and individually controlled by a computer. As will be understood by the skilled artisan upon reading this disclosure, alternative means for producing the braided nanocomposites useful in the electrodes of the present invention can also be used.

The design and analysis of braided structure requires a good understanding of the structural geometry of the braid and the material properties of the constituents. However, these considerations are known to those skilled in braiding. The hierarchical geometry of consists of four structural levels: fiber level, yam level, weave level, and braid level. Additional considerations include molecular orientation and additional phases, for example, carbon nanotube reinforcement at the individual fiber level. The objective will be to account for the material contribution to the stress strain behavior of the braid by taking into consideration of the orientation of the various structural components.

Beginning with a fiber of known or approximated material property (Young's modulus) E_{f}. The fibers, in a multifilament form, are twisted to make yarn of helix angle θ_{y}. These yarns will be braided in a “diamond” braid geometry following a one-up-one-down interlacing pattern with a weave angle θ_{w}. The yarns are braided with a braid angle of θ_{b}. The braided yarns start from a jamming condition with a jamming strain of εⱼ (the strain required to stretch a relaxed braid to a jamming state or the condition under which the oblique diameter of the braiding yarns equals the circumference of the braid).

The relationship of yarn strain, ε_{y}, to fiber strain, ε_{f}, along the yam axis is as follows:

$${ɛ}_{y} = \frac{{ɛ}_{f}}{{Cos}^{2} θ} \qquad ( 1 )$$

where θ is fiber surface helix angle.

The variation of yam modular as a function of fiber modulus and fiber helix angle can be given by:

$${E}_{y} = \frac{{E}_{f} {cos}^{2} θ ( 3 {tan}^{2} θ )}{2 ( {sec}^{2} θ - 1 )} \qquad ( 2 )$$

Further defining the strain in the braid after jamming as ε_{b}*, and a Poisson's ratio \tilde{ν} for the braid, the yarn strain can be related to the braid strain as follows:

ε_{y}ε_{b}*(cos²θⱼ−sin²θⱼ) (3)

where θⱼ is the braid angle at jamming state.

For a Hookean materials it follows that the resistance to tensile deformation of the braid E_{b} can be related to yarn modulus E_{y} by:

E_{b}=E_{y}cos²θⱼ(cos²θⱼ−vsin²θⱼ) (4)

However, materials with different or more complex mechanical properties than these stated here and which are governed by differing equations may also be used to form a part in the invention.

### EXAMPLE 1

Tubular braids comprising 6 and 12 electrodes over glass micropipettes can be prepared. The micropipette base is held in a small chuck or glued to a syringe needle to hold it in the braiding machine.

### EXAMPLE 2

A tungsten form can be partly electrolytically etched from a tungsten rod. The etched tungsten core is stiff enough to penetrate tissue, but is laterally compliant. The tungsten braiding form can be stabilized through the braiding process by longitudinal tension, applied either by holding in a very fine chuck or gluing in place at the distal end while mounting to a hypodermic needle at the proximal end. The braid will be glued to the shaft with cyanoacrylate. After completion of the basic braid, the braid will be freed at the distal end. A probe can be created by electrolysis of the tip of the tungsten rod.

### EXAMPLE 3

The braid process will occur approximately as in Example 2. The tungsten rod will be fully shaped and then held by a fine, for example 9-0, silk thread glued on either end with soluble starch based adhesive. When braid construction is complete the distal braid will be glued to the etched shaft with cyanoacrylate. The assembly will then be soaked in distilled water hanging from the braided wires to free the silk thread which is gently removed from both ends, leaving tungsten and braid structure.

### EXAMPLE 4

The build will proceed as in Example 3, but the distal half of the tungsten shaft will be covered with a polyimide tube to increase diameter. After freeing the assembly, and removing the tube, the braid on the shaft about half-way will be secured with 9-0 suture and the braid gently elongated to near its jammed state. The distal shaft will be led through one of the interstices of the elongated braid and the braid allowed to return to its former state. A tungsten rod length with now protrude from a loosely coiled braid above the 9-0 suture knot, for gripping with microforceps or other attachment to a microdrive.

### EXAMPLE 5

Tubular braid ‘jamming’ and ‘unjamming’ provide a simple way to control stiffness of a cylindrical beam, or stack. The jammed state, like the Chinese finger trap, holds the structure together. Tensioning and untensioning the braid moves from jammed to unjammed states. Deploying such an electrode in vivo, and the change in mechanical properties, may potentially be very rapid, justifying the added complexity of design and construction which are needed for this approach.

This can be accomplished by employing the jamming properties of braids layered over a removable form. Two concentric polyimide tubes, an etched tungsten rod probe, and stainless steel wire rod, and hypodermic tubing can be used to construct the braid. Braid construction will use mannose stabilization of components and tensioning, followed by fixation and dissolving for the final use.

It may be preferable to use a second braid as the jamming system, leaving the wire braid to simply conform to applied forces and changes of the probe. The second braid could also be made of a stiffer but resorbable material in a chronic application.

What is Claimed:

1. A sterilizable electrode comprising a braid and having a plurality of electrically independent conductors, at least some of the conductors being in electrical communication with a plurality of sites on the electrode for sensing or stimulation.

2. The electrode of claim 1 wherein the braid is tubular, flat, or figured.

3. The electrode of claim 1 wherein said configuration has an alterable compliance.

4. The electrode of claim 1 wherein said configuration has an alterable shape.

5. The electrode of claim 1 further comprising a generator wherein the generator send electrical signals to the sites in a pre-selected spatial and temporal pattern.

6. The electrode of claim 1 wherein at least one of the conductors forms part of the braid.

7. The electrode of claim 1 wherein at least one of the conductors is laid into the braid.

8. The electrode of claim 1 wherein at least one conductor is a monofilament.

9. The electrode of claim 1 wherein at least one conductor has a length that is at least 100 times greater than its diameter.

10. The electrode of claim 1 wherein substantially all of the conductors are monofilaments.

11. The electrode of claim 1 wherein at least one conductor is greater than 1 μm in diameter.

12. The electrode of claim 1 wherein substantially all of the conductors are greater than 1 μm in diameter.

13. The electrode of claim 1 wherein substantially all of the conductors are less than 1 μm in diameter.

14. The electrode of claim 1 wherein at least one conductor is less than 100 nm in diameter.

15. The electrode of claim 1 wherein substantially all of the conductors are less than 100 nm in diameter.

16. The electrode of claim 1 wherein at least some of the conductors comprises metal, conductive polymer, conductive protein, or a conductive nanotube or nanofilament.

17. The electrode of claim 1 in which at least a portion of the braid comprises fibroin, keratin, collagen, elastin, poly-L-lactic acid, polylysine, polyethylene oxide, polyaniline, a blend of polyethylene oxide/polyaniline , polyacrylonitrile, poly-ethylene-dioxythiophene, Poly(3,4-ethylenedioxythiophene):poly(styrenesulfonate), poly(3,4-ethylenedioxythiophene)/polyacrylonitrile, polyaniline, or polylactic-co-glycolic acid.

18. The electrode of claim 1 wherein at least one conductor comprises nichrome or stainless steel.

19. The electrode of claim 1 wherein the conductors comprise lithium doped polyanaline.

20. The electrode of claim 1 wherein at least one conductor comprises carbon nanotubes.

21. The electrode of claim 1 further comprising shape memory polymers or shape memory metals

22. The electrode of claim 1 wherein the conductors are insulated with TEFLON® or Parylene-C.

23. The electrode of claim 1 wherein the electrode further comprises a fiberoptic fiber strand

24. The electrode of claim 23 wherein the fiberoptic fiber strand comprises quartz.

25. The electrode of claim 23 wherein the fiberoptic strand is sputter-coated with any metal, gold, platinum, silver, iridium, or combinations thereof.

26. The electrode of claim 1 further comprising a cannula, catheter, or other surgical device

27. The electrode of claim 1 comprising a biodegradable material.

28. The electrode of claim 27 wherein the biodegradable material comprises vicryl, sucrose, dextrose, carbowax, mannose, or polyethylene glycol.

29. The electrode of claim 1 further comprising a material that is dissolvable.

30. The electrode of claim 1 further comprising quantum dots.

31. The electrode of claim 1 wherein at least one conductor is hollow.

32. The electrode of claim 1 wherein said electrode forms at least one triode, tetrode, or polytrode.

33. The electrode of claim 1 wherein said recording or stimulating sites are inside the braid.

34. The electrode of claim 1 wherein said braid forms a part of a composite structure.

35. A sterilizable electrode comprising a plurality of braids and having a plurality of electrically independent conductors, at least some of the conductors being in electrical communication with a plurality of sites on the electrode for sensing or stimulation.

36. The electrode of claim 35 wherein the plurality of braids are arranged in an array.

37. The electrode of claim 35 wherein the plurality of braids are arranged in a cuff configuration.

38. A method of forming a sterilizable electrode comprising:
braiding a plurality of fibers onto, over or within a braiding form to form a braided structure, wherein at least some of the fibers are independently conductive; and
modifying at least some of the fibers to expose them to the environment, such exposure taking place at a plurality of sites;
the fibers being biocompatible and able to withstand sterilization.

39. The method of claim 38 further comprising a plurality of independent conductors being laid into the braid.

40. The method of claim 39 further comprising modifying at least some of the conductors by exposing them to the environment.

41. The method of claim 38 further comprising mechanically altering the compliance of the braid.

42. The method of claim 38 further comprising mechanically altering the shape of the braid.

43. The method of claim 38 wherein the braiding form is hollow.

44. The method of claim 38 wherein said fibers are braiding as a Chinese finger trap.

45. The method of claim 38 further comprising braiding a plurality of shape memory polymers within the braided structure.

46. The method of claim 38 further comprising braiding a plurality of shape memory metals within the braided structure.

47. The method of claim 38 further comprising microelectromechanical systems within the braided structure.

48. The method of claim 38 wherein the braiding form is biodegradable or dissolvable.

49. The method of claim 38 wherein the braiding form has shape memory properties.

50. The method of claim 38 wherein the braiding form is conductive.

51. The method of claim 3 8 wherein the braiding form has an alterable compliance.

52. The method of claim 38 wherein the braided structure is affixed to the braiding form.

53. The method of claim 52 wherein the braided structure is affixed to the braiding form with adhesive.

54. The method of claim 52 wherein the braided structure is affixed to the braiding form with biodegradable or dissolvable material.
